(19) United States
(12) Patent Application Publication
(10) Pub. No.: US 2016/0001001 A1
(43) Pub. Date: Jan. 7, 2016

(54) VENTILATORY ASSIST DEVICE

(71) Applicants: Norbert WRUCK, Lübeck (DE); Stefan MERSMANN, Lübeck (DE); Ernst SCHUBERT, Lübeck (DE); DRÄGER MEDICAL GMBH, Lübeck (DE)

(72) Inventors: Norbert WRUCK, Lübeck (DE); Stefan MERSMANN, Lübeck (DE); Ernst SCHUBERT, Lübeck (DE)

(21) Appl. No.: 14/767,078

(22) PCT Filed: Feb. 11, 2014
(86) PCT No.: PCT/EP2014/000369

§ 371 Date: Aug. 11, 2015

(30) Foreign Application Priority Data
Feb. 13, 2013 (DE) 10 2013 002 408.0

(51) Int. Cl. A61M 5/172; A61M 16/00

(52) U.S. Cl. CPC A61M 5/1723; A61M 16/0069; A61M 2205/3327; A61M 2205/3303; A61M 2230/40

(57) ABSTRACT

A respiratory support device (10) includes a ventilator (20) which allows spontaneous breathing, a control device (30), a dosing device (40) for pharmaceutically active substances, and at least one first measuring device (50). Pharmacodynamic and/or pharmacokinetic data of pharmaceutically active substances and/or compositions as well as comparison data for different respiration parameters are stored in the control device (30). The measuring device (50) is configured to detect one or more respiration parameters. The measuring device (50) or another measuring device is additionally configured such that the effective active ingredient quantity of one or more pharmaceutical substances dispensed by the dosing device can be detected using the measuring device. A device for exchanging data in a bidirectional manner is arranged at least between the control device and the measuring device and/or the dosing device.

### CROSS REFERENCE TO RELATED APPLICATIONS

This application is a United States National Phase Application of International Application PCT/EP2014/000369 filed Feb. 11, 2014 and claims the benefit of priority under 35 U.S.C. §119 of German Patent Application 10 2013 002 408.0 filed Feb. 13, 2013, the entire contents of which are incorporated herein by reference.

### FIELD OF THE INVENTION

The present invention pertains to a ventilatory assist device as well as to a method for operating a ventilatory assist device.

### BACKGROUND OF THE INVENTION

Ventilatory assist devices are generally known. They are used especially when patients have an insufficient spontaneous breathing function. Such a ventilatory assist or even complete mechanical ventilation may require, for example, intubation or tracheotomy of the patient, so that the patient being ventilated requires a drug therapy for establishing a certain absence of pain and frequently also for switching off consciousness.

A large number of analgesics (painkillers), sedatives (tranquilizers) and substances that are used to switch off both pain and consciousness (analgosedation) have been known in this connection. For example, various opioids, e.g., fentanyl, sufentanil, remifentanil, morphine, etc., are used. However, propofol, various benzodiazepines, alpha-2-antagonists or various volatile anesthetics, e.g., desflurane, isoflurane, enflurane, sevoflurane or halothane, may be used as well.

However, many of these substances may have a considerable respiratory depressant effect, which may become noticeable as an adverse side effect especially when the patient is being weaned from the mechanical ventilation or even from the ventilatory assist. When such a respiratory depression is present, the patient is typically no longer able to breathe spontaneously to a sufficient extent, and the efficacy of the gas exchange in the lungs may be compromised to such an extent that there is a threat of the patient suffocating despite the fact that a possibly weak breathing activity is present.

It is currently common practice to reduce the depth of sedation of the patient gradually especially when weaning the patient from mechanical ventilation or from mechanical ventilatory assist. In this way the patient shall get gradually used to breathing again spontaneously until the patient can eventually be extubated. However, it may still be necessary for various medical reasons to continue administering analgesic to the patient.

DE 10 2008 003 237 A1 describes in this connection a device for controlling the depth of sedation. The breathing pattern of a mechanically ventilated patient is determined in this case by means of a tidal volume flow-measuring device integrated in the ventilator (also known as a respirator). The depth of sedation of the patient can then be inferred from this breathing pattern. The depth of sedation over the course of the day can be subsequently changed by means of this device, for example, by means of a control device and a drug dosing device, by correspondingly adjusting the administration of sedatives. In this way, the automatic waking up of a patient at a preset time can be made possible, so that an automatic extubation test can be performed by means of a signal delivered specifically by the control device, for example, before a doctor's round.

EP 2 319 567 A1 provides for a device for controlling a ventilator. To monitor the depth of sedation of a patient, this device has means for monitoring parameters of the CNS. For example, these means may be an electroencephalograph (EEG) or an electromyograph (EMG).

However, it is common to all these prior-art solutions that even though they have the possibility of automatically monitoring the depth of sedation, they lack the possibility of automatically detecting an incipient or developing respiratory depression.

### SUMMARY OF THE INVENTION

Based on this, an object of the present invention is to provide an improved ventilatory assist device.

The device shall be able to be embodied in an especially cost-effective manner and with the simplest means possible.

Furthermore, the device shall be able to offer the possibility of recognizing the risk of development or occurrence of respiratory depression, especially of a drug-induced respiratory depression. Such a respiratory depression shall be able to be taken into account automatically, for example, by means of the device according to the present invention when controlling the device, especially during automatic attempts at waking up and/or during the automatic preparation for extubation attempts within the framework of weaning. The device shall thus be designed such that it can be used as an auxiliary device to determine whether extubation can be performed safely.

In a ventilatory assist device, the present invention makes provisions for the device to have a ventilator which allows spontaneous breathing, a control device, a dosing device for pharmaceutically active ingredients and at least a first measuring device, wherein pharmacodynamic and/or pharmacokinetic data of pharmaceutically active ingredients and/or compositions are stored in the control device, wherein comparison data for various respiration parameters are stored in the control device, and wherein the measuring device is designed such that one or more respiration parameters can be detected by means of the measuring device, and wherein the measuring device or an optionally present, additional measuring device is designed such that the effective active ingredient quantity of one or more of the pharmaceutical ingredients dispensed by the dosing device can be detected, and wherein a bidirectional data exchange means (a bidirectional data exchange connection) is arranged at least between the control device and the measuring device and/or the dosing device.

Ventilatory assist by means of the device according to the present invention comprises both the ventilatory assist specifically as well as complete mechanical ventilation of a patient. Ventilatory assist is defined specifically as the mechanical assistance of spontaneous breathing, when the patient is in a state in which the patient is, in principle, capable of breathing spontaneously, but the gas exchange in the lungs is not sufficient because of the weak breathing activity. Complete mechanical ventilation of a patient is defined as ventilation during which spontaneous breathing is essentially suppressed and during which a check is performed episodically only in the course of weaning, i.e., during the weaning from the ventilatory assist, to determine whether the patient is capable of breathing spontaneously. A device according to the present invention for assisting the ventilation of a patient can thus be able to be used both for the mechanical assisting of spontaneous breathing and for the complete mechanical ventilation of a patient. Therefore, the device for assisting the ventilation of a patient is a device that can be used during weaning, and therefore, it is consequently a device suitable for weaning. Thus, a ventilatory assist device according to the present invention is preferably not an anesthesia device, especially not an anesthesia device for use in an operating room.

A ventilator which allows spontaneous breathing is typically a ventilator that is designed such that a patient, whose breathing is assisted by means of this device, can breath spontaneously within certain limits or even completely. For example, the patient can control the frequency of breaths himself, and the ventilator will then assist the patient only in terms of the depth of breaths by presetting, for example, the tidal volume or the pressure during inspiration. Thus, it may be a device that is suitable for patient-triggered ventilation. For example, the patient can breath in independently. The ventilator which allows spontaneous breathing can recognize this attempt at breathing in and trigger the inspiration (breathing in).

The control device of such a device according to the present invention may have one or more modules for data storage and data processing, which will hereinafter also be called memories. These modules may be connected to one another by a data exchange connection, preferably a bidirectional data exchange connection. The control device may have both one memory or more than one memory. These may both be integrated in the control device or designed as separate components. Measured values detected by means of the measuring device can be inputted into and stored in such a memory. Already known values, e.g., the pharmacokinetic or pharmacodynamic parameters of the desired pharmaceutically active ingredients, may be stored as well. Known values for typical respiration parameters may also be stored in such a memory. In the sense of the present invention, both the known pharmacokinetic or pharmacodynamic parameters and the respiration parameters are values that are called comparison values. In this connection the control device may have only one memory, which is used for both inputting the measured values and storing the comparison values. The control device may have a first memory, which is used to detect and/or store the measured values of the measuring device or measuring devices, and at least one second memory, in which comparison values are stored. In this connection the first memory and the second memory may communicate with one another.

The control device may have, furthermore, a computer. The computer may both be integrated in the control device and designed as a separate component. Such a computer may be used and designed to compare the stored comparison values with the detected measured values. The computer may be connected for this purpose to the memory or memories via a data exchange means, preferably a bidirectional data exchange connection. For example, a decision value can be determined by means of the computer. Based on such a decision value, the computer can then select, for example, a control command. For example, a selection of various control commands may be stored in the control device, for example, in one of the above-mentioned memories or in an another memory. The control device can then select a control command on the basis of a determined decision value and send it to a component of the device according to the present invention, which component is connected to the control device. Such a control command may preferably be able to be selected by means of the computer of the control device. The control command can then be transmitted from the control device to the dosing device, the ventilator or the measuring device by means of the bidirectional data exchange connection. The control command may, of course, also be transmitted at the same time to a plurality of the components mentioned (dosing device, ventilator, measuring device).

The limits may be set concretely and the decision values can be programmed and the control commands can be assigned by the operator of the device according to the present invention. For example, an operator who has a corresponding medical training can input the corresponding values via a correspondingly adapted operation interface before the start of the use of the device according to the present invention. It is recognized in this respect that it is favorable if the device according to the present invention has an operation interface. The control device and/or the computer of the device according to the present invention may therefore be programmable. A selection of decision values and/or control commands, from which the operator of the device can optionally make a selection, may be stored in advance in the control device. It is therefore favorable if a selection of decision values or control commands is stored in the control device for the optional selection by the operator.

The dosing device of the device according to the present invention may be, for example, a device for the intravenous administration of pharmaceutically active ingredients or compositions. The device may be a device for the administration by inhalation of pharmaceutically active ingredients or compositions. The dosing device may be a device that is suitable for the administration of pharmaceutically active ingredients or compositions both intravenously and by inhalation.

A pharmaceutically active ingredient is defined as a compound that can affect the physiological state of a patient, for example, an analgesic, sedative or anesthetic. A pharmaceutically active composition is defined here as a mixture of substances, in which both pharmaceutically active ingredients, for example, analgesics, sedatives or the like, and pharmaceutically inactive ingredients, for example, vehicles for pharmaceutically active ingredients, may be present. Both pharmaceutically active ingredients and pharmaceutically active components are generally also called “drugs” in the present context.

It is common practice that pharmacodynamic data of pharmaceutically active ingredients and/or compositions are defined in the sense of the present invention as data that pertain to the action mechanism of drugs on the body. For example, the pharmacodynamic data stored in the control device may be data that describe the effect of one or more drugs on the CO₂ sensitivity of the respiratory center. It can be recognized, for example, by means of such data when there is a risk that the respiratory center of a patient will not respond to an increase in the CO₂ level in the blood in a physiologically correct manner. The stored pharmacodynamic data may be data that describe the effect of one or more drugs on the 0₂ sensitivity of the respiratory center. It can be recognized in this case by means of the stored data when there is a risk that the respiratory center of a patient will not respond to an oxygen deficiency in the blood in a physiologically correct manner. Furthermore, the stored pharmacodynamic data may be data that describe the effect of one or more drugs on the pH sensitivity of the respiratory center. It can be recognized in this case by means of the stored data when there is a risk that the respiratory center of a patient will not respond to a change in the pH value in the blood in a physiologically correct manner. In all cases, there may be, for example, a relation of certain drug concentrations to certain probability values for the onset of a certain physiological effect.

It is common practice that pharmaceutical data of pharmaceutically active ingredients and/or compositions are defined in the sense of the present invention as data that pertain to the chemical and/or physical processes that lead to a change in the drug concentration in the body and hence to a change in the intensity of the effect of the drug effect. For example, they may be data that provide information on how fast a drug can be metabolized by the body. They may, for example, also be data that provide information on what concentration of a drug may be present in the blood of a patient when a certain concentration of the drug is measured in the breathing gas of the patient and vice versa.

Therefore, pharmacodynamic and/or pharmacokinetic data of pharmaceutically active ingredients and/or compositions in the sense of the present invention may be both pharmacodynamic data of one or more pharmaceutically active ingredients and pharmacokinetic data of one or more pharmaceutically active ingredients, pharmacodynamic data of one or more pharmaceutically active compositions, pharmacodynamic data of one or more pharmaceutically active compositions, pharmacodynamic and pharmacokinetic data of one or more pharmaceutically active ingredients, pharmacodynamic and pharmacokinetic data of one or more pharmaceutically active compositions, or even combinations thereof.

Respiration parameters in the sense of the present invention may be measured values that provide information on the current breathing activity of a patient. Such respiration parameters may also provide information on the quality of the spontaneous breathing of a patient. Examples of such measured values are airway resistance (AR), end-expiratory CO₂ concentration (etCO₂, also called end-tidal CO₂), respiratory minute volume (RMV), partial oxygen saturation (Sp0₂) or oxygen concentration (FiO₂), respiration rate, for example, spontaneous respiration rate (fₛₚₙ), tidal volume (Vₜ), various flow parameters, various pressure levels, without this list being complete by any means. For example, the depth of sedation and the ability to breathe normally spontaneously can also be inferred from such parameters. In particular, the spontaneous respiration rate, tidal volume and end-expiratory CO₂ concentration may now be used as indicators to assess whether a patient is breathing normally. Corresponding comparison data, consequently thus comparison data for respiration parameters, may be stored in the control device for all the respiration parameters mentioned.

A measuring device in the sense of the present invention may be both a measuring device that detects measured blood values, a measuring device that detects measured concentration values in the breathing gas, or a measuring device that detects the tidal volume flow resolved over time, or a measuring device that detects the variability of inspiration and/or expiration times, preferably on the basis of the measured CO₂ concentrations, or a measuring device that detects muscle control processes relevant for breathing. For example, a measuring device in the sense of the present invention may be both a measuring device that detects various respiration parameters or also a measuring device that can analyze measured blood or breathing gas values in respect to a drug that was administered to the patient. For example, it may be possible to determine by means of such a measuring device the concentration at which a certain administered drug is present in a blood sample of a patient. The concentration at which an administered drug is present in a breathing gas sample of a patient may also be determine by means of such a measuring device.

The effective active ingredient quantity of a drug, which is relevant for a patient being ventilated, can be determined on the basis of such measured values. The effective active ingredient quantity is defined in this case as the quantity of a drug that can actually produce an effect on the body. In other words, the effective active ingredient quantity is the concentration of an active ingredient actually prevailing in the body of a patient, i.e., the quantity of the active ingredient that was administered to the patient and was also actually absorbed by the body. It may happen namely for the greatest variety of reasons, for example, especially in case of administration of drugs by inhalation, that the total administered dose cannot be absorbed by the body via the lungs at all. The effective active ingredient quantity of a drug is therefore the quantity of active ingredient that is actually present in the body, especially in the blood of the patient. The effective active ingredient quantity can be determined, for example, by measuring the concentration of an active ingredient or of a metabolic product of the active ingredient in the blood or in the breathing gas of the patient.

At least one bidirectional data exchange connection each is present between the above-described components of the device according to the present invention, namely, between the control device and the measuring device or between the control device and the dosing device or even both between the control device and the measuring device and between the control device and the dosing device. One or more bidirectional data exchange connection may also be present between the control device and the ventilator. Respective bidirectional data exchange connections are preferably present both between the measuring device and the control device and between the dosing device and the control device and between the ventilator and the control device. These bidirectional data exchange connections may be used to enable measured values detected by the measuring device to be transmitted to the control device, settings pertaining to the quantity of drugs dispensed by the dosing device to be transmitted from the dosing device to the control device, values pertaining to the current setting of the ventilator to be transmitted from the ventilator to the control device, and/or control commands to be transmitted from the control device to the dosing device, the ventilator and/or the measuring device. For example, a bidirectional data exchange connection between the control device and a measuring device may be used both to enable the measuring device to transmit measured values from the measuring device to the control device at regular intervals and the control device to request measurements from the measuring device, for example, as a function of determined decision values or control commands. Similarly, a bidirectional data exchange connection between the control device and the dosing device may be used both to transmit control commands from the control device to the dosing device and to transmit feedback on the dispensed quantity of drug or, e.g., also the quantity of drug that is still available and can be dispensed from the dosing device to the control device.

It is seen that it is advantageous if the ventilator has a breathing line. Such a breathing line may have an inspiration line. Such a breathing line may also have an expiration line. The breathing line may have, furthermore, a Y-piece. Both the inspiration line and the expiration line preferably open into the Y-piece. The breathing line may have, furthermore, a patient line. This may likewise be connected to the Y-piece.

The ventilator may have, furthermore, a gas port and/or a room air supply unit, and it may have auxiliary means for disposing of waste gas. Furthermore, the control device may be integrated in the ventilator. However, the control device may be a separate assembly unit of the device.

It is especially advantageous if the dosing device can be controlled by the control device. The control device may control the dosing device by transmitting control commands via the bidirectional data transmission means. The control commands may be able to be selected on the basis of a decision value by the control device, for example, by the computer of the control device. The decision value may be determined, for example, by means of the computer on the basis of measured values that were transmitted from the measuring device to the control device.

As was already described above, the dosing device is, for example, a dosing device for substances (drugs) administered by inhalation and/or a dosing device for intravenously administered substances. The dosing device may be suitable for administration of substances both by inhalation and intravenously. For example, the dosing device may have one or more subunits, which are intended each for the administration of a drug. Thus, a first drug may be administered, e.g., intravenously, while a second drug is administered by inhalation at the same time.

It is seen that it is favorable if the dosing device has at least one drug feed line.

A drug feed line is defined here as a line that can be connected to an adapter, with which a direct or indirect contact can be established with the blood circulation or with the airways of the patient. For example, a drug feed line may be able to be connected to an infusion needle, a breathing mask or a breathing tube. The adapter may also be part a part of the drug feed line. The dosing device of a device according to the present invention may have both drug feed lines that can be connected to the blood circulation and drug feed lines that can be connected to the airways of the patient. It is seen that it is favorable if the drug feed line is a line for drugs administered by inhalation and/or intravenously and if the dosing device has a plurality of drug feed lines.

It is seen, furthermore, that it is favorable if the ventilator can be controlled by the control device.

For example, the control device may transmit selected control commands to the ventilator by means of the bidirectional data transmission means. The control commands may be selected, as described above, on the basis of the determined measured values or the decision value.

Furthermore, it is favorable if the device comprises a measuring arrangement that has a measuring device and at least one second measuring device.

For example, the measuring arrangement may comprise the first measuring device that may be used to detect one or more respiration parameters and a the second measuring device that is used to determine measured values relating to an administered drug from a blood or breathing gas sample. In this connection the device may have a measuring arrangement with more than two measuring devices, for example, the second measuring device may detect measured values from a blood sample, while yet another, third measuring device detects measured values from a breathing gas sample or vice versa. At any rate, it is favorable if all measuring devices of the measuring arrangement of the device are connected to the control device via bidirectional data exchange connection. All measured values of the measuring devices can thus be inputted in the control device in one or more memories and processed by means of the computer.

It is seen that it is, furthermore, favorable if the measuring arrangement the first and/or second measuring device has at least one sensor device. The sensor device is preferably a sensor device for detecting physiological parameters.

Physiological parameters are measured values that can be directly or indirectly derived from a body fluid, the breathing gas or the body surface of a patient. For example, physiological parameters in the sense of the present invention may be the O₂ concentration in the blood, the CO₂ concentration in the blood, the pH value of the blood. The concentration of a certain chemical substance, for example, a drug or a metabolite of a drug in the blood or in another body fluid of the patient may be such a physiological parameter. The concentration of a certain chemical substance, for example, the concentration of a drug, for example, a volatile anesthetic, or of a metabolite of a drug may be such a physiological parameter. A muscle action potential or a neurobiological signal may be such a physiological parameter. For example, it may be a muscle action potential of a muscle relevant for breathing. A neurobiological signal may be, for example, a neuronal action potential of the CNS or a neuronal action potential of a nerve that is relevant for the control of breathing.

A sensor device may be in this connection any device that has a sensor for one of the above-described physiological parameters. The sensor device detects the measured values that will be processed later by the control device in the form of raw data. In this connection the sensor device may be connected to a data processing device of the measuring device, so that the measuring device can process the measured values before these are transmitted to the control device. However, the sensor device may be connected directly to the bidirectional data transmission means, with which the measuring device communicates with the control device, and that the measured values are transmitted as raw data from the sensor device of the measuring device to the control device.

The first measuring device may have a sensor device for detecting physiological parameters, that the second measuring device may have a sensor device for detecting physiological parameters, or both the first measuring device and the second measuring device may have a sensor device each for detecting physiological parameters. The first measuring device, the second measuring device or both measuring devices may have two or more sensor devices each for detecting physiological parameters.

It is seen that it is favorable if the sensor device is a sensor device for detecting respiration parameters.

For example, the sensor device may have a sensor that monitors the breathing gas flow of the patient that is released to the device for ventilation. Such a sensor may be arranged, for example, in the inspiration tube, in the expiration tube, in the Y-piece or in the patient adapter. The sensor may be a volume flow sensor, a differential pressure sensor or an ultrasound sensor. The sensor device may have a plurality of such sensors. A plurality of sensors of the same type or even different sensors may be present in this case.

The sensor device may have a sensor array for detecting a muscle action potential or a superimposition of a plurality of muscle action potentials. Such a sensor array may be positioned, for example, on the skin of the patient being ventilated and designed such that it detects electric or electromagnetic signals that are sent during the activation of neuromuscular synapses.

It is seen, furthermore, that it is favorable if the sensor device is a sensor device for detecting the effective active ingredient quantity of pharmaceutically active ingredients and/or compositions that were administered to the patient.

For example, the sensor device may have a sensor, by means of which the concentration of a chemical substance in a liquid or a gas can be detected. This sensor can detect especially the concentration of volatile anesthetics in the breathing gas. In this way, the sensor can make available, for example, a measured value that describes the degree to which the consciousness is switched off. In case of some active ingredients, for example, enflurane, isoflurane, sevoflurane or desflurane, the measured value may also describe the existing active contribution to the respiratory depression (markedly pronounced) as well as the active contribution to the analgesia (rather weakly pronounced).

This sensor may detect especially the concentration of propofol in the exhaled gas and thus can make available a measured value that describes the extent of respiratory depression and of the switching off of consciousness by propofol. Further, the sensor device may have means that determine a respiration-depressant effect from a measured concentration and a dosing rate analyzed by calculation. This effect may preferably also be displayed by means of the device according to the present invention.

The first measuring device may have a sensor device for detecting the effective active ingredient quantity of pharmaceutically active ingredients and/or compositions, that the second measuring device has a sensor device for detecting the effective active ingredient quantity of pharmaceutically active ingredients and/or compositions, or that both the first measuring device and the second measuring device have a sensor device each for detecting the effective active ingredient quantity of pharmaceutically active ingredients and/or compositions. The first measuring device, the second measuring device or both measuring devices may have each two or more sensor devices for detecting the effective active ingredient quantity of pharmaceutically active ingredients and/or compositions.

The first measuring device may have a sensor device for detecting respiration parameters, while at least one second measuring device has a sensor device for detecting the effective active ingredient quantity of pharmaceutically active ingredients and/or compositions. Furthermore, the first measuring device may have both a sensor device for detecting respiration parameters and a sensor device for detecting the effective active ingredient quantity of pharmaceutically active ingredients and/or compositions. In this connection that the second measuring device also may have both a sensor device for detecting respiration parameters and a sensor device for detecting the effective active ingredient quantity of pharmaceutically active ingredients and/or compositions and that the second measuring device has either a sensor device for detecting respiration parameters or a sensor device for detecting the effective active ingredient quantity of pharmaceutically active ingredients and/or compositions.

It is seen that it is favorable if the measuring arrangement the first and/or second measuring device has at least one data acquisition line.

Such a line may be a connection between a collection device or a receiving device for a sample to be analyzed and the measuring device. The sample to be analyzed is, for example, a body fluid or breathing gas. For example, a collection point for collecting a breathing gas sample may be formed in the area of the breathing tube, i.e., consequently the inspiration tube or even of the expiration tube. The data acquisition line may be a data acquisition line from breathing gas. In this connection the line may be a suction line. Such a suction line can draw the breathing gas sample to be analyzed from the breathing tube, especially from the collection point, and send it to the measuring device. The breathing gas sample can then be analyzed in the measuring device, for example, by means of the sensor device. It is seen that it is favorable if the line has a possibility of connection to the breathing line.

The line may be a data line for a non-suctioning measurement of anesthetic gas. A sensor, which detects the concentration of the volatile anesthetic gas (of the volatile anesthetic) in the breathing gas, is usually arranged in the area of the breathing line in case of such a non-suctioning measurement of anesthetic gas. This sensor is typically connected to a measuring device via the data line. In this connection the line may be such a data line, and the sensor detects a volatile anesthetic, or that the line is such a data line, and the sensor detects a volatile drug, which does not necessarily have to be an anesthetic.

Furthermore, the data acquisition line may be a line for acquiring data from body fluids. Such a line may be connected to a sampling device. A sampling device may be designed in this case such that it can take a sample of a body fluid to be analyzed. A sampling device may also be able to be connected to the patient directly or indirectly. For example, the sampling device may be a cuvette or a similar receiving device, into which a body fluid can be filled. The sampling device may be an infusion needle or the like, which can be connected to the patient. At any rate, the data acquisition line may be connected to a sensor device. The data acquisition line has a first end, which faces the measuring device, and a second end, which faces the sampling device. The sensor device may be arranged at the first end, for example, at or in the measuring device. The sensor device may also be arranged at the second end, for example, at or in the sampling device. The sampling device may be able to be connected to the patient, so that the data acquisition line can also be connected to the patient at least indirectly, e.g., via a cannula or a breathing gas line. It is therefore seen that it is favorable if the data acquisition line can be connected to the sensor device and/or to the patient.

It is therefore seen that a ventilatory assist device according to the present invention, which has a ventilator which allows spontaneous breathing, a control device, a dosing device for pharmaceutically active ingredients and at least one first measuring device, wherein at least one bidirectional data exchange connection is arranged between the control device, the measuring device, the dosing device and/or the ventilator, is preferably designed such that both the dosing device and the ventilator can be controlled by the control device. It is favorable if the device also has at least one second measuring device, wherein all measuring devices are connected to the control device via bidirectional data exchange connection. The measuring devices are preferably equipped, as was described above, with one or more sensor devices, as well as optionally with sampling devices and lines for data acquisition. Furthermore, it is useful in such a device if the dosing device and the ventilator are also connected to the control device via the bidirectional data exchange connection. The control device can then control the dosing device and the ventilator on the basis of the measured values that can be determined by means of the measuring devices.

It is seen that it is always advantageous in a device according to the present invention if the control device has means for determining a decision value on the basis of the measured values detected by the measuring device. The means for determining a decision value may be formed, for example, in the computer of the control device.

It is seen, furthermore, that it is favorable if the control device has means for selecting a control command. The means for selecting a control command may also be formed in the computer of the control device. The means for determining a decision value and the means for selecting a control command are preferably designed such that they can communicate with one another.

It is also always advantageous if the control device is designed to transmit selected control commands to the ventilator, the dosing device and/or the measuring device. For example, the control device may be connected to the ventilator via one of the above-described bidirectional data exchange connection, and it can transmit a control command to the ventilator by means of the bidirectional data exchange connection. At the same time that the control device may then receive information on the current operating state of the ventilator via this bidirectional data exchange connection.

The control device may also be connected to the dosing device via one of the above-described bidirectional data exchange connection, and it can transmit a control command to the dosing device by means of the bidirectional data exchange connection. At the same time the control device may then receive information on the current operating state of the dosing device via this bidirectional data exchange connection.

Furthermore, the control device may be connected to a measuring arrangement that comprises one measuring device or a plurality of measuring devices via one of the above-described bidirectional data exchange connections, and the control device can transmit a control command to the measuring device(s) by means of the bidirectional data exchange connection(s). At the same time the control device may then receive information on the current operating state of the measuring device as well as the measured values detected by the measuring device via this bidirectional data exchange connection.

Therefore, the present invention makes provisions, furthermore, for a method for operating a device according to the present invention to comprise the following steps:
a. Automatic detection of respiration parameters by means of a measuring device of the device;
b. Forwarding of the respiration parameters detected to the control device by means of the bidirectional data exchange connection;
c. Automatic detection of the effective concentration of one or more drugs by means of a measuring device of the device;
d. Forwarding of the effective drug concentration value detected to the control device by means of the bidirectional data exchange connection;
e. Selection of at least one control command in the control device; and
f. Forwarding of the control command to the ventilator and/or to the dosing device by means of the bidirectional data exchange connection.

The device may be designed, for example, such that it has a measuring device, which is designed such that it can detect both one or more of the above-described respiration parameters and an effective drug concentration. However, as was already described above, the device have a measuring arrangement that comprises a plurality of measuring devices. For example, a first measuring device can detect the respiration parameters in step a, while a second measuring device detects the effective drug concentration in step c. However, of course, the measuring arrangement may be only one measuring device, with which both steps a and c can be carried out. At any rate, the detection of the effective drug concentration during the carrying out of the method is defined such that the absolute concentration of a pharmaceutically active ingredient in a body fluid sample or in a sample of the breathing gas flow of a patient is detected by the measuring device with which step c is carried out. The measuring device may optionally determine the above-described effective active ingredient quantity from this absolute concentration. However, the measuring device may transmit only the absolute concentration value to the control device. The control device can then determine the effective active ingredient quantity thereafter. The effective concentration of a drug in the sense of the method according to the present invention may therefore be both the absolute concentration of a drug in a body fluid or breathing gas sample and the above-described effective active ingredient quantity.

It is seen in this connection that it is favorable if step a is carried out with a first measuring device for respiration parameters and that step c is carried out with a second measuring device for measuring the effect of the drug, which is different from the first measuring device. This is especially favorable when the measuring device for measuring the drug effect is a measuring device which shall analyze a body fluid sample. For example, the detection of the effective drug concentration may contain the automatic performance of a quantitative immunochemical, spectroscopic, chromatographic or other specific test.

Furthermore, it is advantageous if the steps a and c are carried out simultaneously. In addition, it is favorable if steps b and d are carried out simultaneously. However, it is not necessary in the sense of a simultaneous performance to perform steps a and c in a time-synchronized manner, and, in particular, it is not necessary to start and end the steps a and c in a time-synchronized manner. It is rather sufficient if there is a common time period within which the steps in question are carried out independently from one another at any desired time. This also applies to steps b and d. In particular, it is not necessary for steps a and c or b and d to be carried out in a certain sequence. The steps a, b, c and d may be carried out independently from one another in any desired sequence, but step b is only carried out if step a had been performed before at any desired time, and step d is only performed if step c had been performed before at any desired time.

It is seen, furthermore, that it is favorable if the selection of the control command in step e comprises the step of
e.1 Determining a decision value in the control device, and step e.1 optionally comprises one or more of the following steps:
e.2 Comparison of the detected respiration parameter from step a and/or of the detected effective drug concentration from step c with data that are stored in the memory device of the control device; and
e.3 Forwarding of the detected respiration parameter from step a and/or of the detected effective drug concentration from step c and/or of the detected decision value to an output device.

The decision value may be, for example, a certain concentration of a drug in a body fluid or in a breathing gas sample of a patient. The decision value may also be the determined effective active ingredient quantity. The decision value may be a respiration parameter. At any rate, information that can be processed by the computer of the control device may be assigned to the data detected by the measuring devices and transmitted to the control device. This is regardless of whether it is an absolute concentration, already processed information, e.g., the effective active ingredient quantity, a characteristic for a respiration parameter, e.g., pressure, CO₂ concentration, pH value or the like. This information can then be compared with the data processed in the control device. This information can then be compared with the comparison data stored in the memory or memories for respiration parameters and/or with the stored pharmacodynamic and/or pharmacokinetic data. Depending on whether the determined value is greater or lower than or equal to a value from the pool of the stored data, a corresponding decision value is assigned to the information supplied by the measuring device, i.e., the determined respiration parameter or the determined effective drug concentration. This decision value may then be simply outputted, for example, by an output unit. For example, the output unit may be a monitor, printer, alarm system or another output device. A monitor can then display to the operator, for example, the operator of the device, that a certain decision value is available, and the operator can determine what actions he would like to take next on the basis of this decision value. The output may be carried out by printing or by transmitting an alarm signal, for example, to a nurses' station or the like.

It is also favorable if a control command can be selected in the device on the basis of the determined decision value. For example, one or more control commands, which may be assigned to certain decision values, may be stored in the control device. The control device can then select a control command on the basis of the decision value, for example, by the computer comparing the decision value or decision values with corresponding stored data.

It may be also favorable in this connection if the determination of the decision value in step e.1 comprises the following steps:

I. Comparison of the determined respiration parameters with data that are stored in a memory of the control device;
ii. Comparison of the determined effective drug concentration with data that are stored in a memory of the control device;
iii. Determination of the risk of development of a drug-induced respiratory depression and/or determination of the extent of the potential respiration-depressant effect of the administered drug on the basis of the comparison performed in step ii; and
iv. Determination of a decision value on the basis of the data obtained in steps I and iii.

Further features, details and advantages of the present invention appear from the text of the claims as well as from the following description of exemplary embodiments on the basis of the drawings and from the further examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic diagram of a first exemplary embodiment of a device according to the present invention;

FIG. 2 is a schematic diagram of another exemplary embodiment of a device according to the present invention;

FIG. 3 is a schematic diagram of another exemplary embodiment of a device according to the present invention;

FIG. 4 is a schematic diagram of another exemplary embodiment of a device according to the present invention; and

FIG. 5 is an example of a chronological sequence of the ventilation of a patient by means of a device according to the present invention.

### DESCRIPTION OF THE PREFERRED EMBODIMENTS

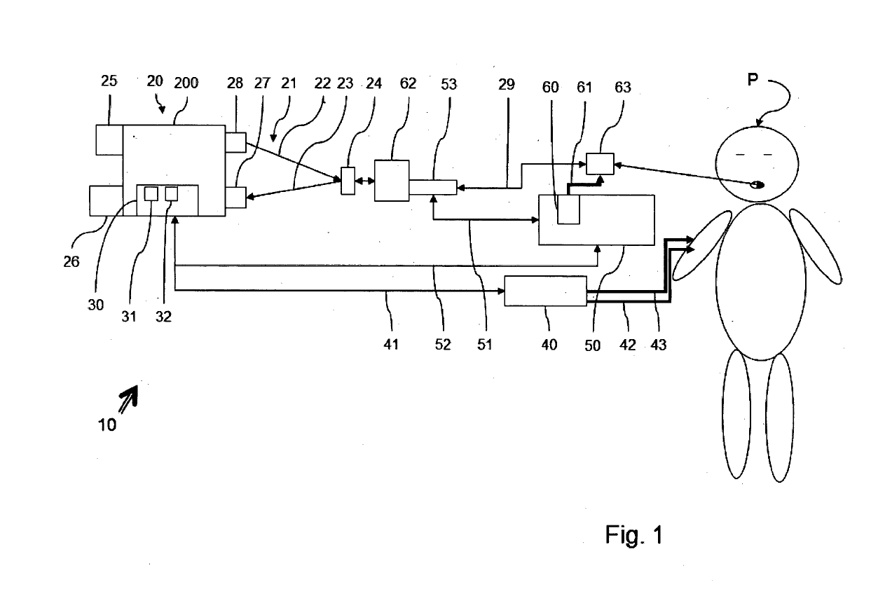

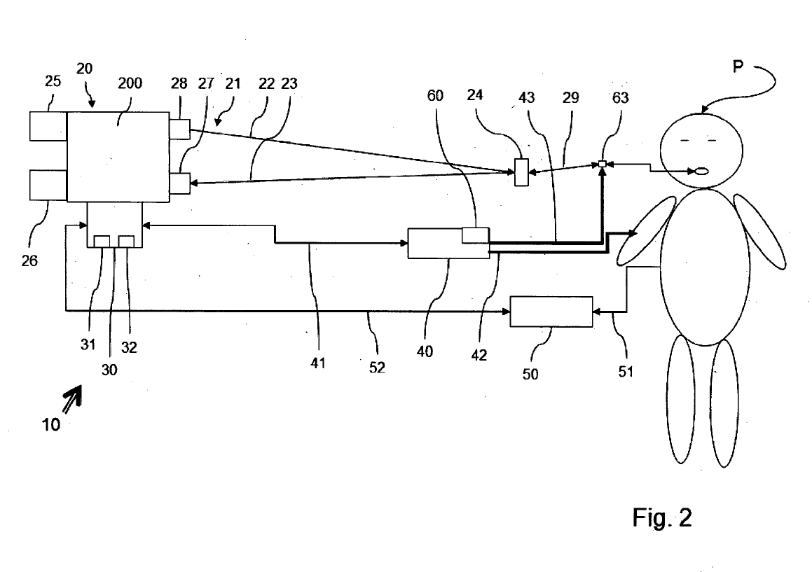

A schematic view of a device 10 according to the present invention is seen in FIG. 1. The device 10 has, just like the devices 10 shown schematically in FIGS. 2, 3 and 4, a ventilator 20 which allows spontaneous breathing with a control device 30, a dosing device 40 and a measuring arrangement comprised of a measuring device 50. Both the dosing device 40 and the measuring device 50 are connected to the control device 30 by means of bidirectional data exchange connection 41, 52. A patient P can be ventilated or the breathing of the patient P can be assisted by means of the device 10. At the same time, different drugs can be fed to the patient P intravenously or by inhalation.

Just like the ventilator 20 shown in FIGS. 2, 3 and 4, the ventilator 20 in FIG. 1 contains a ventilator unit 200 controllable by means of the control device 30 and has a breathing line 21. The breathing line 21 has an inspiration line 22, an expiration line 23, a Y-piece 24 and a patient line 29. The inspiration line 22 is connected at one of its ends to the ventilator unit 200 via a connection piece 28 and at its other end to the Y-piece 24. The expiration line 23 is connected at one of its ends to the ventilator line 200, likewise via a connection piece 29, and at its other end to the Y-piece 24. The patient line 29 is likewise connected to the Y-piece. The patient line 29 is, for example, a ventilation tube. This may be connected to an adapter (not shown), for example, a breathing mask. However, it may also be an intubation tube.

Just like the ventilator 20 shown in FIGS. 2, 3 and 4, the ventilator 20 in FIG. 1 has, furthermore, a gas port 25. Fresh breathing air can be fed to the ventilator 20 by means of this gas port 25. A breathing air source (not shown) can be connected to the gas port 25. It may be a compressed air source or even a simple inlet for room air.

In addition, the ventilator 20 in FIG. 1 has, just like in FIGS. 2, 3 and 4, a waste gas outlet 26. The air flowing back from the breathing line 21 can be released into the surrounding area through this waste gas outlet 26. A disposal device (not shown), which removes drug residues that may possibly be present from the air flowing back, may be connected to the waste gas outlet 26.

The control device 30 is integrated in the ventilator 20 in the example shown. The ventilator unit 200 and the control device 30 may be accommodated for this simply in a common housing. Just like the control device 30 shown in FIGS. 2, 3 and 4, the control device 30 in FIG. 1 has a memory 31 and a computer 32. A plurality of memories 31 may, of course, be present as well.

The dosing device 40 is a device for dosing drugs that can be administered intravenously. In the example shown, the device 40 has a first drug feed line 42 for a first drug and a second drug feed line 43 for a second drug. The device 40 may have only one drug feed line or more than two drug feed lines. The dosing device 40 is connected to the control device 30 via the bidirectional data transmission means in the form of a bidirectional data transmission connection 41. The dosing device 40 can be connected either directly to the patient or to an adapter, for example, an infusion cannula (infusion needle) via the drug feed lines 42, 43.

The device 10 has, furthermore, a drug evaporator 60. The drug evaporator 60 is connected to a port 63 for feeding the drug via a drug feed line 61. The port 63 is arranged on or in the patient line 29, so that a volatile drug, which is dispensed from the drug evaporator 60, can be fed to the breathing line 21. The patient P can therefore inhale and then exhale such a volatile anesthetic via the breathing line 21. To collect exhaled drug gas, the device 10 has, furthermore, a drug gas intermediate storage unit 62. The volatile drug collected therein can be inhaled by the patient P again during his next breath. The patient P can, furthermore, also exhale metabolized forms or even excess quantities of, for example, an intravenously administered drug. As a consequence, certain concentrations of a drug administered via the dosing device 40 may also be detectable as drug residues in the breathing gas. These drug residues are also collected in the drug gas intermediate storage unit 62. The effective active ingredient quantity can be determined from the concentration of these drug residues.

A sampling device 53, which is arranged in the breathing line 21, as is shown, preferably in the patient line 29, is provided for checking the concentration of such a drug residue, volatile drug, the metabolized form of a volatile drug in the breathing gas or the like. A sensor, which detects the concentration of the desired substance, is arranged in this sampling device 53. Furthermore, sensors, which detect various other respiration parameters, namely, airway resistance (RR), end-expiratory CO₂ concentration (etCO₂), tidal volume (RMV), partial oxygen saturation (SpO₂), respiration rate or the like, may be arranged here as well. The sampling device 53 is connected to the measuring device 50 via a data acquisition line 51. The measuring device 50 is connected, in turn, to the control device 30 via a bidirectional data exchange connection 52. At any rate, the measuring device 50 is designed such that it can detect both the effective active ingredient quantity of one or more administered substances and one or more of the respiration parameters described here.

The embodiment variant of the device 10 according to the present invention, which is shown schematically in FIG. 2, also has, as was already described above in connection with FIG. 1, a ventilator 20 which allows spontaneous breathing, a control device 30, a dosing device 40 and a measuring device 50.

The dosing device 40 shown in FIG. 2 is connected to the control device 30 via a bidirectional data exchange connection 42. The dosing device 40 also has a first drug feed line 42 and a second drug feed line 43. The first drug feed line 42 is a drug feed line for drugs that can be administered intravenously. The first drug feed line 42 can be connected to an adapter, for example, an infusion needle. The first drug feed line 42 may also be connected directly to the patient. The second drug feed line 43 is a drug feed line for volatile drugs, which can be fed with the breathing gas flow. The second drug line 43 is connected to a port 63 for feeding the drug. The port 63 is arranged in the patient line 29. The dosing device 40 has, furthermore, a drug evaporator 60 for providing the volatile drug. The drug evaporator 60 may be arranged in a common housing with the dosing device 40 or be a separate assembly unit.

The measuring device 50 shown in FIG. 2 is connected to the control device 30 via a bidirectional data exchange connection 52. Furthermore, the measuring device 50 has a data acquisition line 51. The measuring device 50 is designed in this case such that it can analyze a body fluid of a patient. The data acquisition line 51 is connected to an adapter (not shown). The adapter may be connected either directly to the patient P or to a sampling device (likewise not shown). A sample of a body fluid can be transported into the measuring device 50 via the data acquisition line 51. A sensor device can then detect the desired measured value in the measuring device 50. As an alternative, the data acquisition line 51 may also be connected to a sensor device arranged outside the measuring device 50, for example, to a measuring electrode for muscle action potentials or an ECG electrode. At any rate, the measuring device 50 is designed such that it can detect both the effective active ingredient quantity of one or more administered substances and one or more of the respiration parameters described here.

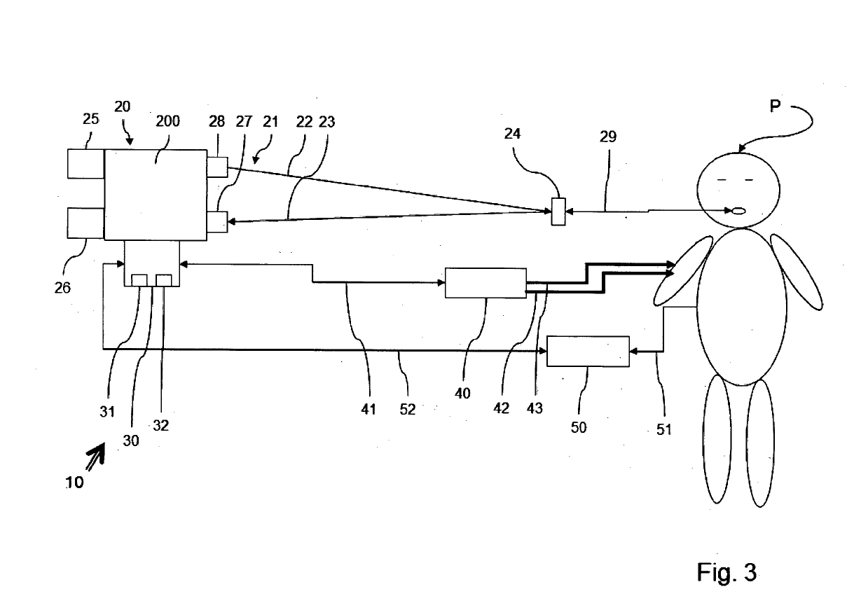

The embodiment variant of the device 10 according to the present invention, which is shown schematically in FIG. 3, also has, as was already described above in connection with FIG. 1, a ventilator 20 which allows spontaneous breathing, a control device 30, a dosing device 40 and a measuring device 50. The control device 30 is shown here as an example as an external component of the ventilator 20. It may, of course, also be integrated in the ventilator 20.

As was likewise described in connection with FIG. 1, the dosing device 40 is a device for dosing drugs that can be administered intravenously. In the example shown, the device 40 has a first drug feed line 42 for a first drug and a second drug feed line 43 for a second drug. The device 40 may of course have only one or a plurality of drug feed lines. The dosing device 40 is connected to the control device 30 via the bidirectional data transmission means 41. The dosing device 40 can be connected via the drug feed lines 42, 43 either directly to the patient or to an adapter, for example, an infusion cannula.

The measuring device 50 shown in FIG. 3 is connected to the control device 30 via a bidirectional data exchange connection 52. Furthermore, the measuring device 50 has a data acquisition line 51. The measuring device 50 is designed in this case such that it can analyze a body fluid of a patient. The data acquisition line 51 is connected to an adapter (not shown). The adapter may be connected either directly to the patient P or to a sampling device (likewise not shown). A sample of a body fluid can be transported into the measuring device 50 via the data acquisition line 51. A sensor device can then detect the desired measured value in the measuring device 50. As an alternative, the data acquisition line 51 may also be connected to a sensor device arranged outside the measuring device 50, for example, a measuring electrode for muscle action potentials or an ECG electrode. At any rate, the measuring device 50 is designed such that it can detect both the effective active ingredient quantity of one or more administered substances and one or more of the respiration parameters described here.

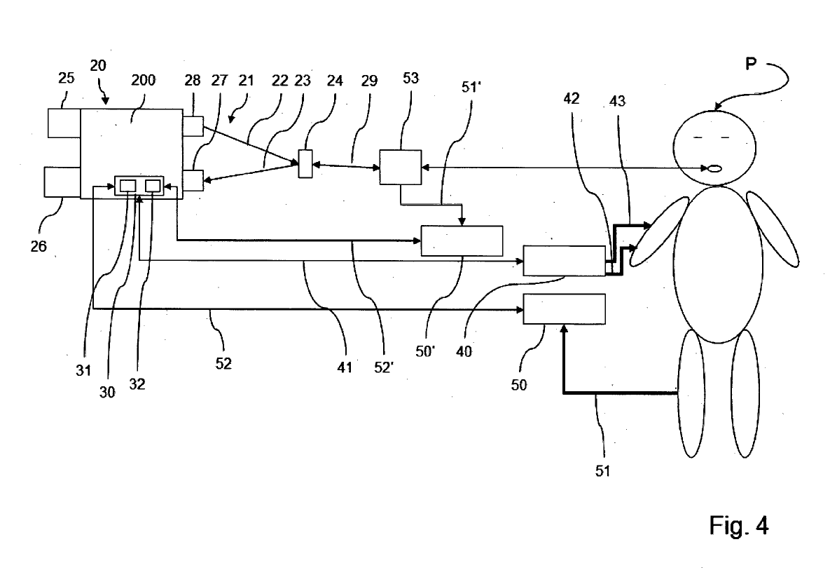

The embodiment variant of the device 10 according to the present invention, which is shown schematically in FIG. 4, also has, as was already described above in connection with FIG. 1, a ventilator 20 which allows spontaneous breathing, a control device 30, a dosing device 40 and a measuring arrangement comprising a measuring device 50. The control device 30 is shown in this case as an example as an external component of the ventilator 20. It may, of course, also be integrated in the ventilator 20. As was already described in connection with FIG. 1 and FIG. 3, the dosing device 40 is a device for dosing drugs that can be administered intravenously. In the example shown, the device 40 has a first drug feed line 42 for a first drug and a second drug feed line 43 for a second drug. The device 40 may of course have only one or a plurality of drug feed lines. The dosing device 40 is connected to the control device 30 via the bidirectional data transmission means 41. The dosing device 40 can be connected via the drug feed lines 42, 43 either directly to the patient or to an adapter, for example, an infusion cannula.

The device 10 according to the present invention shown in FIG. 4 has, the measuring arrangement comprises in addition to the first measuring device 50, a second measuring device 50′. Like the measuring device 50 already described in connection with FIGS. 2 and 3, the first measuring device 50 is connected to the control device 30 via a bidirectional data exchange connection 52. Furthermore, the measuring device 50 has a data acquisition line 51. The measuring device 50 is designed here such that it can analyze a body fluid of a patient. The data acquisition line 51 is connected to an adapter (not shown). The adapter may be connected either directly to the patient P or to a sampling device (likewise not shown). A sample of a body fluid can be transported into the measuring device 50 via the data acquisition line 51. A sensor device can then detect the desired measured value in the measuring device 50. As an alternative, the data acquisition line 51 may also be connected to a sensor device arranged outside the measuring device 50, for example, a measuring electrode for muscle action potentials or an ECG electrode. At any rate, the measuring device 50 is designed such that it can detect both the effective active ingredient quantity of one or more administered substances and one or more of the respiration parameters described here.

The second measuring device 50′ is connected to the sampling device 53 by a line 51′ and to the control device 30 by a bidirectional data exchange connection 52′.

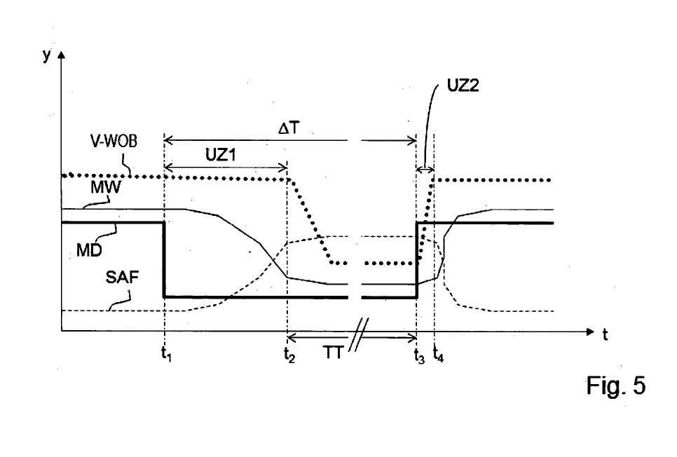

FIG. 5 shows the schematic chronological sequence of a breathing exercise, which can be carried out by means of a device according to the present invention. The device described in FIGS. 1, 2, 3 and 4 is operated using the above-described method for operating the device. The patient can be weaned from mechanical ventilation or ventilatory assist by means of such a breathing exercise. At the same time, it can be observed whether the risk of respiratory depression is present if the patient shall, e.g., be extubated.

FIG. 5 shows four different curves, V-WOB, MW, MD, SAF, which extend along the time axis t, namely, the percentage of the work of breathing that is provided by the ventilator, hereinafter called the ventilator work of breathing V-WOB; the drug effect curve MW; the drug dosing MD, and the curve describing the patient's ability to breath spontaneously, SAF. The percentage of the work of breathing that is provided by the ventilator, i.e., the work of breathing of the ventilator V-WOB, is reciprocal to the patient's lung function (not shown). Axis y of the schematic diagram represents a fictitious number axis in which, as is common practice, higher numerical values are arranged at the top and lower numerical values at the bottom.

Four times t₁, t₂, t₃ and t₄, namely, the start time t₁ of the drug reduction, the start of the breathing exercise t₂, the nominal end of the breathing exercise t₃, which corresponds to the end of the drug reduction, and the effective end of the breathing exercise t₄ are shown on the time axis t. The drug dose administered is reduced for the period between the start time t₁ and the nominal end of the breathing exercise t₃, which can be recognized on the basis of the drug dosing curve MD. The drug dosing curve MD drops at the start time t₁ to a markedly reduced level. The drug dose administered is reduced at this time. The drug dosing curve MD rises again to the previous level at the nominal end of the breathing exercise t₃. Consequently, the drug dose administered is raised again at this time. Therefore, there is a period of drug reduction ΔT between the start time t₁ and the nominal end of the breathing exercise t₃.

Corresponding to the drug dose administered, the effect of the drugs administered is at first high before the start time t₁ and then drops slowly after the start time t₁, as is recognized from the drug effect curve MW. The breathing exercise t₂ starts as soon as the effect of the drugs administered has dropped to a preset level. The drug effect increases again as a consequence of the repeated increase in drug dosing after the nominal end of the breathing exercise t₃.

Before the start time t₁, the patient is in a state in which the patient is either fully or partially ventilated and in which his own ability to breathe spontaneously is markedly reduced by the effect of the drugs administered and by other external effects. This is seen from the SAF curve describing the patient's ability to breath spontaneously. This is at a low level before the start time t₁ and rises slowly starting from the start time t₁ until the start of the breathing exercise t₂. The ability to breathe spontaneously starts to drop again slowly with the nominal end of the breathing exercise t₃ as a consequence of the repeated increase of drug dosing. This drop accelerates with the effective end of the breathing exercise t₄, after which the SAF curve describing the ability to breathe spontaneously drops again to a low level.

The work of breathing of the ventilator, V-WOB, shows that a large percentage of the work of breathing is assumed by the ventilator before the start time t₁. This percentage also remains constant until the start of the breathing exercise t₂. The work of breathing of the ventilator, V-WOB, drops to a lower level with the start of the breathing exercise t₂. This means that the ventilator assumes a lower percentage of the work of breathing and the percentage of the work of breathing that must be performed by the patient is increased. The work of breathing of the ventilator, V-WOB, increases again to the original level between the nominal end of the breathing exercise t₃ and the effective end of the breathing exercise t₄. This means that the ventilator will again assume a higher percentage of the work of breathing with the resumption of a higher drug dosing and the reduced ability to breath spontaneously, which can be expected as a consequence.

It is seen in FIG. 5 that a first transition period UZ1 is located between the start time t₁ and the start of the breathing exercise t₂ and that there is a second transition period UZ2 between the nominal end of the breathing exercise t₃ and the effective end of the breathing exercise t₄. The period of nominal breathing exercise TT is located between the start of the breathing exercise t₂ and the end of the nominal breathing exercise t₃, i.e., between the first transition period UZ1 and the second transition period UZ2.

The drug dosing curve MD is at a low level during the first transition period UZ1, i.e., a reduced drug dose is administered. The drug effect curve MW drops slowly during this transition period UZ1. This drop is monitored by means of the device 10 according to the present invention, which is shown in FIGS. 1, 2, 3 and 4. In particular, the effective drug concentration is detected by means of a measuring device 50, 50′ corresponding to step c of the method according to the present invention for operating the device 10. At the same time, the SAF curve describing the ability to breathe spontaneously rises during the transition period UZ1. This rise is likewise monitored with the device 10 shown in FIGS. 1, 2, 3 and 4. In particular, the respiration parameters are detected by means of a measuring device 50, 50′ corresponding to step a of the method according to the present invention for operating the device 10. Both the detected respiration parameters and the detected effective drug concentrations are processed in the control device 30 of the device 10, and, as was described above, decision values are determined. The start of the breathing exercise t₂ can be set on the basis of these decision values. The control device 30 can thus transmit a control command to the ventilator 20 at this time, so that the ventilator 20 reduces the ventilation or ventilatory assist of the patient until the level desired during the period of the nominal breathing exercise TT is reached.

The first transition period UZ1 is followed by the period of the nominal breathing exercise TT. The patient's ability to breathe spontaneously is suppressed only slightly or not at all during this period. The effective quantity of the drugs administered with a respiration-depressant side effect is correspondingly adjusted. The device 10 described in FIGS. 1, 2, 3 and 4 detects the respiration parameters and the effective drug concentration during this period as well by means of the measuring device or measuring devices 50, 50′.

The second transition period UZ2 starts with the end of the nominal breathing exercise t₃. The drug dose is raised again at this time, so that the drug dosing curve MD rises again. The percentage of the lung function that is assumed by the ventilator 30 is also increased again until the end time t₄, so that the ventilator work of breathing curve V-WOB also rises again during the transition period UZ2.

### Example of Stored Respiration Parameters

Examples of respiration parameters that may be stored in the device 10 according to the present invention, in particular in the control device 30 according to the above-described exemplary embodiments are, e.g.,

Spontaneous respiration rate (fₛₚₙ)

Tidal volume (Vₜ)
as well as

End-tidal CO₂ (etCO₂).

These respiration parameters include corresponding tolerance ranges, which are taken into account when generating control commands. A respiration rate in the range of 15-30 per minute is usually considered to be normal, and a respiration rate of, e.g., 35 per minute is usually considered to be tachypnea requiring treatment in adults. For example, the following exemplary values listed in Table 1, which are considered to be normal ventilation under certain marginal conditions, may be stored as benchmark data for respiration parameters in the device.

TABLE 1
Meanings of the marginal conditions: 0 = no known past illness,
1 = neurological disorder known, 2 = COPD patient.
Furthermore, bpm = number of breaths per minute.

| | Fₛₚₙ | Fₛₚₙ | Vₜ | etCO₂ | |
|---|---|---|---|---|---|
| Body | lower | upper | lower | upper | |
| weight | limit | limit | limit | limit | Marginal |
| [kg] | [bpm] | [bpm] | [mL] | [mmHg] | condition |
| 35 to 55 | 15 | 30 | 250 | 55 | 0 |
| 35 to 55 | 15 | 30 | 250 | 65 | 2 |
| 35 to 55 | 15 | 34 | 250 | 55 | 1 |
| >55 | 15 | 30 | 300 | 55 | 0 |
| >55 | 15 | 30 | 300 | 65 | 2 |
| >55 | 15 | 34 | 300 | 55 | 1 |

### Example of the Determination of a Decision Value and the Selection of a Control Command

Tolerance fields are set in the multidimensional respiration parameter space to determine decision values. This means that the control device has means that are designed to associate the measured values determined by the measuring device on the basis of stored data. For example, values for the end-tidal CO₂ partial pressure (etCO₂) and for the spontaneous respiration rate f, may be stored in the control device. It can be set, for example, for the end-tidal CO₂ partial pressure that the control device selects a first decision value El when the actual value drops below a value of 20 mmHg, but it selects a decision value E2 when the actual value drops below a value of 55 mmHg but there is a value of 20 mmHg or higher, and a decision value E3 when the actual value exceeds a value of 55 mmHg. Corresponding decision values can also be assigned to other respiration parameters in the same manner. For example, a decision value E4 may be assigned to a value of 35 bpm or higher for the spontaneous respiration rate fₛₚₙ, but a decision value E5 may be assigned to an f, value of less than 35 bpm but at least 30 bpm, a decision value E6 may be assigned to an fₛₚₙ value of less than 30 bpm but at least 15 bpm, and a decision value E7 may be assigned to an fₛₚₙ value of less than 15 bpm.

It is apparent that this example is only an example and completely different values may, of course, also be stored in the control device.

The selection of the control commands is then performed on the basis of the decision values determined. Both individual decision values and combinations of decision values may be decisive. If, for example, the combination of decision value E5 and decision value E2 was determined in the control device, the corresponding control command may be a command that instructs the ventilator to raise the pressure by a preset value, e.g., 2 mbar or less. Another example would be the determination of the decision values E6 and E2. The corresponding control command may now be a command that instructs the ventilator to lower the pressure by, e.g., 4 mbar or less.

### Examples of Stored Pharmacokinetic Data

The time-dependent courses of the effective drug concentration can be calculated in advance for many relevant drugs by means of models from medical research. For example, an adequate value for the transition time UZ1 can be determined on the basis of the time in which the effective concentration of opioids drops to half the value when the supply is stopped. For example, concentration limits can be assigned in the control device for certain opioids to certain time values. The time values may indicate here the duration of the transition period UZ1. These values may be assigned to a certain body weight or other data of the patient. When using the device, the operator may, for example, program this assignment freely or even select from different suggestions stored in the control device. The duration of the period UZ2 can be determined in the same manner on the basis of stored data. The duration of time during which the full effect of the drug is reached after administration of a certain dose of a drug is important for the control of the device during the period UZ2.

These times of some important drugs used in analgosedation are listed as examples in the table below. The onset time designates the start of the effect after administration of the active ingredient.

TABLE 2
Time elapsing until the maximum analgesic effect is reached

| | | Time elapsing until |
|---|---|---|
| | | the maximum analgesic |
| Drug | Onset time | effect is reached |
| Remifentanil | 1 minute | 2 minutes |
| Sufentanil | 2 minutes | 3 minutes |
| Morphine | 8 minutes | 25 minutes |
| Fentanyl | 3 minutes | 5 minutes |
| Alfentanil | 1 minute | 2 minutes |

### Examples of Stored Pharmacodynamic Data

One example of stored pharmacodynamic data is the interaction that, for example, propofol and remifentanil may have when they are administered together. The risk for the development of respiratory depression can be estimated on the basis of the known administered doses of the drugs.

Respiratory depression reaches its maximum within 5 minutes following the single-time bolus injection of 1 μg/kg of remifentanil and persists for about 10 minutes, and it persists for about 20 minutes following administration of 2 μg/kg. The blood gases return to normal within 5-15 minutes, regardless of the rate of infusion, after a continuous infusion had been stopped.

Infusion rates targeting an effective concentration of 2.0 ng/mL are not unusual in case of intense pain.

These relationships can be used to determine the degree of respiratory depression as a function of time from measured effective quantities or effective quantities determined in another manner, which are consequently known quantities. This can be done by means of pharmacological models; as an alternative, an operator of the device according to the present invention may preset a variation range of the dosage based on his experience. The control of the device according to the present invention uses these data.

All the features and advantages appearing from the claims, the specification, including the following examples and the drawings, including design details, arrangements in space and process steps, may be essential for the present invention both in themselves and in the many different combinations.

While specific embodiments of the invention have been shown and described in detail to illustrate the application of the principles of the invention, it will be understood that the invention may be embodied otherwise without departing from such principles.

1. A ventilatory comprising:
a ventilator which allows spontaneous breathing;
a control device;
a dosing device for pharmaceutically active ingredients; and
at least one measuring device, wherein:
pharmacodynamic and/or pharmacokinetic data of pharmaceutically active ingredients and/or compositions are stored in or accessed by the control device
comparison data for various respiration parameters are stored in or accessed by the control device;
the measuring device is designed such that one or more respiration parameters can be detected by means of the measuring device;
the at least one measuring device or an additional measuring device is designed such that an effective active ingredient quantity of one or more pharmaceutical substances dispensed by the dosing device can be detected with the least one measuring device or the additional measuring device, and
a bidirectional data exchange connection at least one of between the control device and the measuring device and the control device and the dosing device.

2. A device in accordance with claim 1, wherein the dosing device is controlled by the control device.

3. A device in accordance with claim 1, wherein the dosing device has at least one drug feed line.

4. A device accordance with claim 1, wherein the ventilator is controlled by the control device.

5. A device in accordance with claim 1, further comprising at least an additional measuring device.

6. A device in accordance with claim 1, wherein the at least one measuring device has at least one sensor device.

7. A device in accordance with claim 6, wherein the at least one sensor device is a sensor device for detecting respiration parameters.

8. A device in accordance with claim 1, wherein at least one sensor device is a sensor device for detecting at least one of the effective active ingredient quantity of pharmaceutically active ingredients and compositions, which were administered to the patient.

9. A device in accordance with claim 1, wherein the at least one measuring device has at least one data acquisition line.

10. A device in accordance with claim 1, wherein the control device has means for determining a decision value on the basis of the measured values detected by the measuring device.

11. A device in accordance with claim 1, wherein the control device has means for selecting a control command.

12. A device in accordance with claim 11, wherein the control device is designed to transmit selected control commands to the ventilator, the dosing device and/or the measuring device.

13. A ventilatory assist method comprising the steps of:
providing a ventilatory assist device comprising a ventilator for spontaneous breathing, a control device with access to comparison data for various respiration parameters and with access to pharmacodynamic and/or pharmacokinetic data of pharmaceutically active ingredients and/or compositions, a dosing device for providing pharmaceutically active ingredients, a measuring arrangement configured for detecting one or more respiration parameters and for detecting an effective active ingredient quantity of one or more pharmaceutical substances dispensed by the dosing device and a bidirectional data exchange connection at least one of between the control device and the measuring arrangement and between the control device and the dosing device;
automatically detecting respiration parameters with the measuring arrangement;
forwarding the detected respiration parameters to the control device with the bidirectional data exchange connection;
automatically detecting an effective concentration of one or more drugs with the measuring arrangement;
forwarding the detected effective drug concentration to the control device with the bidirectional data exchange connection;
selecting at least one control command with the control device; and
forwarding the control command to at least one of the ventilator and to the dosing device with the bidirectional data exchange connection.

14. A method in accordance with claim 13, wherein:
the measuring arrangement comprises a first measuring device for detecting respiration parameters and a second measuring device for detecting a drug effect;
the step of automatically detecting respiration parameters is carried out with the first measuring device for detecting respiration parameters; and
the step of automatically detecting an effective concentration of one or more drugs is carried out with the second measuring device for detecting the drug effect, which is different from the first measuring device for detecting respiration parameters.

15. A method in accordance with claim 13, wherein the steo pf selecting the control command comprises the step of
determining a decision value in the control device.

16. A method in accordance with claim 15, wherein the step of determining a decision value in the control device comprises one or more of the following steps:
comparing at least one of the detected respiration parameter and the detected effective drug concentration from step with data that are stored in a data storage device of the control device; and
forwarding at least one of the detected respiration parameter and the detected effective drug concentration and the determined decision value to an output device.

17. A ventilatory assist device comprising:
a ventilator for spontaneous breathing;
a control device with access to comparison data for various respiration parameters and with access to pharmacodynamic and/or pharmacokinetic data of pharmaceutically active ingredients and/or compositions;
a dosing device for providing pharmaceutically active ingredients;
a measuring arrangement configured for detecting one or more respiration parameters and for detecting an effective active ingredient quantity of one or more pharmaceutical substances dispensed by the dosing device; and
a bidirectional data exchange connection at least one of between the control device and the measuring arrangement and between the control device and the dosing device, wherein:
the measuring arrangement automatically detects respiration parameters and forwards the detected respiration parameters to the control device via the bidirectional data exchange connection;
the measuring arrangement automatically detects an effective concentration of one or more drugs and forwards the detected effective drug concentration to the control device via the bidirectional data exchange connection;
the control device selects at least one control command and forwarding the selected control command to at least one of the ventilator and the dosing device via the bidirectional data exchange connection.

18. A ventilatory assist device in accordance with claim 17, wherein:
the measuring arrangement comprises a first measuring device for detecting respiration parameters and a second measuring device for detecting a drug effect;
the first measuring device automatically detects respiration parameters; and
the second measuring device automatically detects an effective concentration of one or more drugs for detecting the drug effect; and
first measuring device is different from the second measuring device.

19. A ventilatory assist device in accordance with claim 17, wherein the control device selects at least one control command by determining a decision value in the control device including:
comparing at least one of the detected respiration parameter and the detected effective drug concentration from step with data that are stored in a data storage device of the control device; and
forwarding at least one of the detected respiration parameter and the detected effective drug concentration and the determined decision value to an output device.

20. A ventilatory assist device in accordance with claim 19, wherein:
the dosing device is controlled by the control device; and
the dosing device comprises a drug feed line.
